(19) United States
(12) Patent Application Publication
(10) Pub. No.: US 2021/0289923 A1
(43) Pub. Date: Sep. 23, 2021

(54) COLOR MIXING SYSTEM FOR COSMETIC COMPOSITION

(71) Applicant: Amorepacific Corporation, Seoul (KR)

(72) Inventor: Choonbok JEONG, Yongin-si, Gyeonggi-do (KR)

(73) Assignee: Amorepacific Corporation, Seoul (KR)

(21) Appl. No.: 17/261,354

(22) PCT Filed: Jul. 24, 2019
(86) PCT No.: PCT/KR2019/009163

§ 371 Date: Jan. 19, 2021

(30) Foreign Application Priority Data
Aug. 9, 2018 (KR) 10-2018-0092760

(51) Int. Cl. A45D 44/00; G06Q 30/06; A61B 5/00; A61B 5/103; A61Q 1/02; A61K 8/18

(52) U.S. Cl. CPC A45D 44/005; G06Q 30/0621; A61B 5/441; A45D 2044/007; A61Q 1/02; A61K 8/18; A61B 5/1032

(57) ABSTRACT

One aspect of the present disclosure relates to a color mixing system for a cosmetic composition and, more specifically, to a cosmetic color mixing system enabling the colors of a cosmetic composition to be mixed according to various skin colors of consumers by using a color mixing pigment formulation in which a pigment for mixing the colors of a cosmetic composition into a desired color is manufactured so as to facilitate quality and inventory control.

### TECHNICAL FIELD

This application claims the benefits of priority based on Korean Patent Application No. 10-2018-0092760 filed on Aug. 9, 2018, the entire contents of which are incorporated herein by reference.

One aspect of the present disclosure is relates to a color mixing system for a cosmetic composition capable of preparing a cosmetic composition having colors matching various skin conditions and preferences of consumers in a store where consumers directly purchase cosmetics.

### BACKGROUND ART

Cosmetics are divided into base make-up cosmetics applied on an entire face and point make-up cosmetics applied only on a small part of a face. Base make-up cosmetics are designed to cover freckles, wrinkles, spots and the like of a face to express clean skin, and among point make-up cosmetics, a lipstick is designed to make lips more beautiful, an eye shadow to make eyes beautiful, and a blusher to make it look beautiful by giving brightness of a shade around cheeks.

Thus, color is the most important factor to look beautiful, and pigments are applied to a formulation to provide appearance and color expression.

As cosmetics industry has grown and consumer needs have diversified recently, demands on make-up cosmetic products with more subdivided and standardized color tones have increased.

Accordingly, since there is a variation for each pigment, commercially available cosmetics are developed and produced while managing pigments by number, and diverse and broad demand spectra of customers tend to be satisfied in a manner of diversifying the number.

Make-up cosmetics having a color tone are generally manufactured using a production process of mixing a pigment base (PB) that is a raw material to obtain the defined color tone of the make-up cosmetics product. Herein, colors are diversely expanded based on PB compositions different from each other, and a color for each of the corresponding numbers (No. 21, No. 23 and the like) is adjusted by comparing with a standard product during production to provide a product having a constant color to customers.

However, such a method produces specific colors and thereby has a limit in reflecting skin characteristics of individuals, and a manner of providing foundation with various colors to customers using a method of, while modularizing a make-up composition to a certain color (for example, white, yellow, red, black), standardizing each of the modules to have a constant color in the modularization considering variations in the raw pigments, and then mixing these compositions in a store using a device is proposed.

### PRIOR ART DOCUMENT

(Patent Document 1) U.S. Pat. No. 5,945,112

### DISCLOSURE

### Technical Problem

As a result of extensive studies in view of the above, the inventors of one aspect of the present disclosure have developed a color mixing system for a cosmetic composition, the system provided with, in order to color match a cosmetic composition customized for various skin colors of consumers, a skin measurement module, a control module determining a mixing ratio of pigment formulations for color matching according to the measured skin color measurement value, and a mix module capable of color matching a cosmetic composition by mixing the pigment formulations for color matching according to the simulated mixing ratio. Such a color mixing system for a cosmetic composition is capable of color matching a cosmetic composition suitable for consumer needs and various skin colors and is thereby effective in satisfying specific preference of each consumer.

Accordingly, an aspect of one aspect of the present disclosure provides a cosmetic composition and a color mixing system capable of providing a consumer-customized cosmetic composition.

### Technical Solution

According to an aspect of one aspect of the present disclosure, there is provided a color mixing system for a cosmetic composition, the system including (a) a skin measurement module measuring a skin color of a person subject to skin measurement; (b) a control module simulating, based on data on the measured skin color, a mixing ratio of pigment formulations for color matching capable of obtaining the skin color; and (c) a mix module mixing the pigment formulations for color matching according to the simulated mixing ratio.

The skin measurement module may be one or more types selected from the group consisting of a camera, a visible light spectrum measurement device and a color difference meter.

The data may be one or more types selected from the group consisting of Lab values, RGB values and spectrum values for each wavelength for a skin color.

The pigment formulation for color matching may be a pigment formulating a mixed pigment composition prepared by mixing raw pigments to a cosmetic composition form.

The raw pigment may be one or more types selected from the pigment group consisting of white (W), yellow (Y), red (R), black (B), green (G), blue (B), and pink (P).

The mixed pigment composition may be a standard pigment prepared by mixing the raw pigments and a raw bulking agent, and a standard bulking agent.

The standard pigment and the standard bulking agent, which are the mixed pigment composition, may be mixed using a defined mixing ratio for color matching.

The pigment formulation for color matching may have one or more types of formulations selected from the group consisting of an emulsion, a cream, an oil, a gel, a balm, and an essence.

The raw pigment uses four types of raw pigments of white (W), yellow (Y), red (R), and black (B) when color matching a base make-up cosmetic composition,

a color matching process is conducted by deriving MW (Module White), MY (Module Yellow), MR (Module Red), MB (Module Black), and MK (Module bulKing) values using an inverse matrix in the following Equation 1, and

for each repeated color matching process, types and mixing ratios of the mixed raw pigments may be derived based on MW, MY, MR, MB, and MK:

$$( \begin{matrix}{W}_{W} & {W}_{Y} & {W}_{R} & {W}_{B} & 0 \\ {Y}_{W} & {Y}_{Y} & {Y}_{R} & {Y}_{B} & 0 \\ {R}_{W} & {R}_{Y} & {R}_{R} & {R}_{B} & 0 \\ {B}_{W} & {B}_{Y} & {B}_{R} & {B}_{B} & 0 \\ {K}_{W} & {K}_{Y} & {K}_{R} & {K}_{B} & 100\end{matrix} ) \times ( \begin{matrix}MW \\ MY \\ MR \\ MB \\ MK\end{matrix} ) = ( \begin{matrix}W \\ Y \\ R \\ B \\ K\end{matrix} ) \qquad [ Equation 1 ]$$

In the equation,

W, Y, R, B, and K respectively mean amounts of four types of raw pigments of white, yellow, red, and black and one type of raw bulking agent used,

MW, MY, MR, MB, and MK respectively mean, as a mixed pigment composition, amounts of four types of standard pigments of white, yellow, red, and black and one type of standard bulking agent used,

W_{W}, Y_{W}, R_{W}, B_{W}, and K_{W} respectively mean mixing ratios of white, yellow, red, and black raw pigments and a raw bulking agent used for forming a white standard pigment (MW),

W_{Y}, Y_{Y}, R_{Y}, B_{Y}, and K_{Y} respectively mean mixing ratios of white, yellow, red, and black raw pigments and a raw bulking agent used for forming a yellow standard pigment (MY),

W_{R}, Y_{R}, R_{R}, B_{R}, and K_{R} respectively mean mixing ratios of white, yellow, red, and black raw pigments and a raw bulking agent used for forming a red standard pigment (MR), and

W_{B}, Y_{B}, R_{B}, B_{B}, and K_{B} respectively mean mixing ratios of white, yellow, red, and black raw pigments and a raw bulking agent used for forming a black standard pigment (MR).

W_{W}, Y_{W}, R_{W}, B_{W}, and K_{W} may be respectively from 70 to 96, from 0.1 to 10, from 0.1 to 10, from 0.01 to 5 and from 3 to 7,

W_{Y}, Y_{Y}, R_{Y}, B_{Y}, and K_{Y} may be respectively from 1 to 30, from 60 to 95, from 1 to 30, from 0.1 to 15 and from 1 to 15,

W_{R}, Y_{R}, R_{R}, B_{R}, and K_{R} may be respectively from 1 to 30, from 1 to 30, from 60 to 95, from 0.1 to 15 and from 1 to 15, and

W_{B}, Y_{B}, R_{B}, B_{B}, and K_{B} may be respectively from 1 to 40, from 1 to 20, from 1 to 20, from 40 to 90 and from 1 to 15.

The cosmetic composition is specialized for a base make-up cosmetic composition, and may be one or more types selected from the group consisting of eye make-up and lip make-up by varying a pigmentation composition ratio.

The bulking agent may include one or more types selected from the group consisting of polymethyl methacrylate (PMMA), silica, polymethylsilsesquioxane, an HDI/trimethylol hexyllactone crosspolymer, alumina, talc, sericite, and boron nitride.

### Advantageous Effects

A color mixing system for a cosmetic composition according to one aspect of the present disclosure allows customers to directly color match a cosmetic composition in a store according to various skin colors of the consumers by combining combined pigment formulations for color matching at a specific ratio instead of preparing and selling pigments for color matching a cosmetic composition to a target color by each number that is already matched.

In addition, the color mixing system for a cosmetic composition is capable of adding simplicity to quality and inventory managements by managing pigment formulations for color matching prepared in a form of readily conducting quality and inventory managements.

In addition, when using the color mixing system for a cosmetic composition, a skin color of a consumer is directly measured, and then a cosmetic composition having a color suitable for the measured skin color can be color matched, and as a result, a customized cosmetic composition can be color matched and prepared.

In addition, a method of sampling preparation in a store can be used as well in order to improve satisfaction of a customized product, and through this, customers can directly find colors suited for themselves in a store. In addition, increasingly improved customized formulations can be provided by accumulating such skin color data and color matching data of formulations.

### DESCRIPTION OF DRAWINGS

FIG. 1 is a mimetic diagram illustrating an operation process of a cosmetic color matching system according to the embodiment of present invention.

### BEST MODE

Hereinafter, one aspect of the present disclosure will be described in more detail in order to illuminate one aspect of the present disclosure.

Terms or words used in the present specification and the claims are not to be interpreted limitedly to common or dictionary meanings, and shall be interpreted as meanings and concepts corresponding to technological ideas of the present disclosure based on a principle in which the inventors may suitably define the concepts of terms in order to describe the invention in the best possible way.

A term “raw pigment” used in the present specification means a pigment supplied from a manufacturer. Even the same raw pigment of the same manufacturer may vary in quality depending on environment or time during production and supply. The raw pigment may also be referred to as a pigment base, and when color matching a cosmetic composition according to one aspect of the present disclosure, examples of the raw pigment particularly used as a cosmetic composition for base make-up include four types of white (W), yellow (Y), red (R), and black (B), however, various raw pigments may be used depending on the case.

A term “raw bulking agent” used in the present specification is an additive mixed to prevent, when preparing a standard pigment as to describe later by mixing the raw pigments, agglomeration of the raw pigments.

A term “standard pigment” used in the present specification means, in order to compensate disadvantages of the raw pigment exhibiting quality variability, a pigment exhibiting quality constancy prepared by mixing raw pigments and a raw bulking agent (K) for preventing agglomeration of the raw pigments. The standard pigment may also be referred to as a mix pigment. When color matching a cosmetic composition according to one aspect of the present disclosure, examples of the standard pigment particularly used as a cosmetic composition for base make-up include four types of standard white (MW), standard yellow (MY), standard red (MR), and standard black (MB), however, various standard pigments may be used depending on the case.

A term “standard bulking agent” used in the present specification is an additive mixed to prevent agglomeration of the standard pigments when color matching a cosmetic composition by mixing the standard pigments in a defined ratio.

The term “raw bulking agent” and “standard bulking agent” used in the present specification may refer to the same bulking agent, but may be referred as a raw bulking agent and a standard bulking agent, respectively, in order to distinguish the bulking agent depending on the pigment types used therewith.

A term “pigment formulation for color matching” used in the present specification is a pigment having a specific formulation prepared using the “standard pigment”, and is a basic block of formulation obtained by preparing the “standard pigment” into a formulation and allowing preparation of various final formulations just by simply mixing these. The pigment formulation for color matching may be typically an emulsion.

A color mixing system for a cosmetic composition according to one aspect of the present disclosure may be a device itself for preparing a customized cosmetic composition.

Composition Color Mixing System for a Cosmetic Composition

One aspect of the present disclosure relates to a color mixing system for a cosmetic composition capable of color matching a customized cosmetic composition having colors suitable for skin colors of cosmetic consumers.

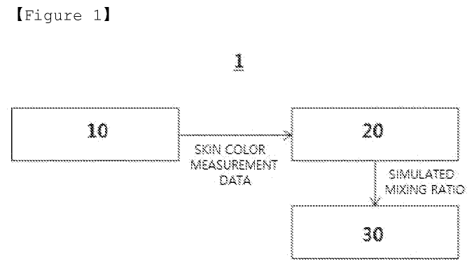

FIG. 1 is a mimetic diagram illustrating an operation process of a cosmetic color matching system according to one aspect of the present disclosure.

When referring to FIG. 1, the color mixing system for a cosmetic composition (1) according to one aspect of the present disclosure may include (a) a skin measurement module (10) measuring a skin color of a person subject to skin measurement; (b) a control module (20) simulating, based on data on the measured skin color, a mixing ratio of pigment formulations for color matching capable of obtaining the skin color; and (c) a mix module (30) mixing the pigment formulations for color matching according to the simulated mixing ratio.

In one aspect of the present disclosure, the skin measurement module is a device for measuring a skin color of a person subject to skin measurement, and may be one or more types selected from the group consisting of a camera, a visible light spectrum measurement device and a color difference meter, but is not limited thereto as long as it is a device capable of measuring a skin color. Preferably, the skin measurement module may be a camera, and when using a camera, results of the skin measurement may change by an external light source, and a skin color of a person subject to skin measurement may be measured by simultaneously photographing a means employed as a base of a skin color such as a standard color chip.

Data on skin colors may be obtained using the skin measurement module. Herein, the obtainable skin color data may be one or more types selected from the group consisting of, for example, Lab values, RGB values and spectrum values for each wavelength. L is luminosity that is brightness, and a combination means complementary color of green to red and b combination means complementary color of yellow to blue.

In one aspect of the present disclosure, the control module may simulate, based on the skin color data obtained with the skin measurement module, a mixing ratio of pigment formulations for color matching capable of obtaining the skin color.

The control module may be formed including an input unit for inputting skin color data obtained with the skin measurement module; a calculation unit for determining a mixing ratio of a mixed pigment composition based on the skin color data; and an output unit for delivering the simulated mixing ratio to a mix module to describe later.

The pigment formulation for color matching is a pigment obtained by formulating a mixed pigment composition prepared by mixing raw pigments into a cosmetic composition form, and may be formulated into one or more types selected from the group consisting of an emulsion, a cream, an oil, a gel, a balm, and an essence, but is not limited thereto, and may be diversely formulated into possible formulation forms that may be marketed as cosmetics.

In one aspect of the present disclosure, the mixed pigment composition may be prepared using raw pigments and a raw bulking agent (K) for preventing agglomeration of the raw pigments.

As the raw pigment, identical products are supplied from a manufacturer, however, there are variations in quality for each supplied raw pigment lot depending on environment and time during production and supply. In addition, even when conducting a color matching process using the same raw pigment lot, there may be variations in the color of the color matched cosmetic composition for each process. In other words, even when color matching the cosmetic composition with the same raw pigment using the same process, the raw pigment varies in quality when environment and time are different during production and supply of the raw pigment, and a color of the cosmetic composition color matched using the raw pigment may vary as well.

Accordingly, a mixed pigment composition exhibiting quality constancy may be prepared so as to compensate a disadvantage of the raw pigment exhibiting variations in quality, and used instead of the raw pigment.

In the present invention, when the cosmetic composition to mix is for base make-up, a mixed pigment composition may be prepared by combining white (W), yellow (Y), red (R), and black (B) as the raw pigment.

As the mixed pigment composition in one aspect of the present disclosure, various mixed pigment compositions may be prepared depending on the types and the mixing ratios of the mixed raw pigments among white (W), yellow (Y), red (R), and black (B), which are the raw pigments.

When preparing the mixed pigment composition, the raw pigments are in a powder form and tend to agglomerate, and a bulking agent may be mixed together in order to prevent agglomeration of the raw pigments, and the bulking agent used when mixing with the raw pigments is referred to as a raw bulking agent.

In one aspect of the present disclosure, only four types of standard pigments are illustrated as the mixed pigment composition, however, as described above, additional standard pigments may also be prepared in addition to the four types of standard pigments depending on the types and the mixing ratios of the raw pigments. In one aspect of the present disclosure, the four types of standard pigments are referred to as standard white (MW), standard yellow (MY), standard red (MR), and standard black (MB).

The four types of standard pigments may be prepared by mixing the four types of raw pigments using different raw pigment types and mixing ratios. Herein, agglomeration may occur when mixing just the four types of raw pigments, and a raw bulking agent may be mixed together in order to prevent this.

In addition, in one aspect of the present disclosure, the mixed pigment composition may be prepared by milling. A raw bulking agent is used in order to prevent agglomeration of the raw pigments for preparing the mixed pigment composition, however, product quality of the mixed pigment composition may be improved when mixing the raw pigments and the bulking agent by milling.

Herein, the raw bulking agent may include one or more types selected from the group consisting of polymethyl methacrylate (PMMA), silica, polymethylsilsesquioxane, an HDI/trimethylol hexyllactone crosspolymer, alumina, talc, sericite, and boron nitride.

In addition, the raw bulking agent may have a spherical or powder form.

Meanwhile, although the raw bulking agent may be used in order to prevent agglomeration of the raw pigments when the mixed pigment composition is prepared by milling, an oil without the powder type bulking agent may be used instead of the bulking agent to prevent raw pigment agglomeration. Herein, the oil may be one or more types selected from the group consisting of a silicone-based oil, an ester-based oil, and a hydrocarbon-based oil, but is not limited thereto, and oils that may be used in the art to prevent powder agglomeration may be properly used depending on compatibility.

As the milling, millings used in the art may all be used, and preferably, a roll mill may be used.

In general, a cosmetic composition is diversely defined in the color depending on the skin tone or preference of a user. For example, in the case of a cushion foundation that is a base make-up product, the color is defined as No. 21, No. 23 and the like depending on the skin tone of a user.

When preparing a cushion foundation composition using the mixed pigment composition, that is, four types of standard pigments and one type of standard bulking agent, the amounts of the four types of standard pigments and the one type of standard bulking agent required to prepare the cushion foundation composition are fixed.

Since the mixed pigment composition always has constant quality, a cosmetic composition always having the same quality may be color matched when mixing the mixed pigment composition, that is, four types of standard pigments, in a defined ratio. Herein, a bulking agent may be mixed together when mixing the four types of standard pigments in a defined ratio in order to prevent agglomeration of the standard pigments, and the bulking agent used when mixing with the standard pigments is referred to as a standard bulking agent.

For example, when color matching a No. 21 cushion foundation composition using the mixed pigment composition, a mixing ratio of the mixed pigment composition for preparing the No. 21 cushion foundation composition may be defined as a fixed value. Since the mixed pigment composition also has the same quality, a No. 21 cushion foundation composition always having the same quality may be color matched just by mixing the mixed pigment compositions using the following mixing ratio when color matching the No. 21 cushion foundation composition is required.

In one aspect of the present disclosure, when color matching a cosmetic composition using the mixed pigment composition, actual amounts of the used raw pigments may be accurately calculated reducing inventory of the raw pigments, and as a result, costs for storage or disposal required for inventory of the raw pigments may be reduced.

In one aspect of the present disclosure, when color matching a cosmetic composition using the mixed pigment composition, MW, MY, MR, MB, and MK values may be obtained using an inverse matrix in the following Equation 1. Accordingly, even when using raw pigments with different manufacturers, different time of production, or with the same manufacturer but having variations in quality by a lot unit, a cosmetic composition having the same quality may be color matched regardless of manufacturer, time of production or a lot of the raw pigments using just a process of obtaining MW, MY, MR, MB, and MK values by adding or subtracting types and amounts of the raw pigments to mix.

In addition, only a case of using four raw pigments is illustrated in the following Equation 1, however, this illustrates only a case of color matching a cosmetic composition for base make-up, and as necessary, a case of using more types of raw pigments such as a cosmetic composition for color make-up may also be added to the following Equation 1.

$$( \begin{matrix}{W}_{W} & {W}_{Y} & {W}_{R} & {W}_{B} & 0 \\ {Y}_{W} & {Y}_{Y} & {Y}_{R} & {Y}_{B} & 0 \\ {R}_{W} & {R}_{Y} & {R}_{R} & {R}_{B} & 0 \\ {B}_{W} & {B}_{Y} & {B}_{R} & {B}_{B} & 0 \\ {K}_{W} & {K}_{Y} & {K}_{R} & {K}_{B} & 100\end{matrix} ) \times ( \begin{matrix}MW \\ MY \\ MR \\ MB \\ MK\end{matrix} ) = ( \begin{matrix}W \\ Y \\ R \\ B \\ K\end{matrix} ) \qquad [ Equation 1 ]$$

In the Equation,

W, Y, R, B, and K respectively mean amounts of four types of raw pigments of white, yellow, red, and black and one type of raw bulking agent used,

MW, MY, MR, MB, and MK respectively mean, as a mixed pigment composition, amounts of four types of standard pigments of white, yellow, red, and black and one type of standard bulking agent used,

W_{W}, Y_{W}, R_{W}, B_{W}, and K_{W} respectively mean mixing ratios of white, yellow, red, and black raw pigments and a raw bulking agent used for forming a white standard pigment (MW),

W_{Y}, Y_{Y}, R_{Y}, B_{Y}, and K_{Y} respectively mean mixing ratios of white, yellow, red, and black raw pigments and a raw bulking agent used for forming a yellow standard pigment (MY),

W_{R}, Y_{R}, R_{R}, B_{R}, and K_{R} respectively mean mixing ratios of white, yellow, red, and black raw pigments and a raw bulking agent used for forming a red standard pigment (MR), and

W_{B}, Y_{B}, R_{B}, B_{B}, and K_{B} W_{B}, Y_{B}, R_{B}, B_{B}, and K_{B} respectively mean mixing ratios of white, yellow, red, and black raw pigments and a raw bulking agent used for forming a black standard pigment (MR).

In addition, W_{W}, Y_{W}, R_{W}, B_{W}, and K_{W}, parameters defined for preparing the standard pigment in Equation 1, may be respectively from 70 to 96, from 0.1 to 10, from 0.1 to 10, from 0.01 to 5 and from 3 to 7. When the parameters are outside the above-mentioned range, it may be difficult to obtain a white color with quality suitable for color matching a cosmetic composition.

W_{Y}, Y_{Y}, R_{Y}, B_{Y}, and K_{Y} may be respectively from 1 to 30, from 60 to 95, from 1 to 30, from 0.1 to 15 and from 1 to 15. When the parameters are outside the above-mentioned range, it may be difficult to obtain a yellow color with quality suitable for color matching a cosmetic composition.

W_{R}, Y_{R}, R_{R}, B_{R}, and K_{R} may be respectively from 1 to 30, from 1 to 30, from 60 to 95, from 0.1 to 15 and from 1 to 15. When the parameters are outside the above-mentioned range, it may be difficult to obtain a yellow color with quality suitable for color matching a cosmetic composition.

W_{B}, Y_{B}, R_{B}, B_{B}, and K_{B} may be respectively from 1 to 40, from 1 to 20, from 1 to 20, from 40 to 90 and from 1 to 15. When the parameters are outside the above-mentioned range, it may be difficult to obtain a black color with quality suitable for color matching a cosmetic composition.

As described above, the mixed pigment composition is a standard pigment standardized in white, yellow, red, and black so as to color match colors of already commercialized cosmetic compositions. However, when used in the color mixing system for a cosmetic composition according to one aspect of the present disclosure, an optimal mixing ratio may be calculated so as to color match a cosmetic composition suitable for a skin color of a consumer based on skin color data secured by directly measuring a skin color of the consumer, and as a result, a customized cosmetic composition suitable for various skin colors of consumers may be color matched by mixing in the above-mentioned mixing ratio.

In addition, for readiness during management and cosmetic composition preparation, the mixed pigment composition may be used in the color mixing system for a cosmetic composition according to one aspect of the present disclosure after being prepared to the pigment formulation for color matching formulated in various formulations as described above.

Using the mixing ratio of the pigment formulations for color matching delivered from the control module, the mix module of the cosmetic composition according to one aspect of the present disclosure is capable of color matching a consumer-customized cosmetic composition using a simple process of homogeneously mixing the pigment formulations for color matching.

Herein, the mix module may include a mixer for mixing the pigment formulations for color matching.

Specifically, the mix module may include a vessel holding the pigment formulations for color matching, a nozzle transporting the pigment formulations for color matching to a mixer, a control device allowing the mixing ratio of the pigment formulations for color matching simulated in the control module to be transported to the mixer that is a mixer mixing the pigment formulations for color matching, and a control unit controlling transport and mixing of the pigment formulations for color matching.

Examples of the mixer may include an agi mixer, however, the mixer is not limited thereto, and mixers used for mixing cosmetic compositions in the art may be widely used.

In one aspect of the present disclosure, the cosmetic composition to color match may be selected from the group consisting of a base make-up cosmetic composition, an eye make-up cosmetic composition, and a lip make-up cosmetic composition.

The base make-up cosmetic composition may be one or more types selected form the group consisting of cushion foundation, powder foundation, liquid foundation, cream foundation, and sun cream.

The eye make-up cosmetic composition may be one or more types selected form the group consisting of eye shadow, eye liner, and mascara.

The lip make-up cosmetic composition may be one or more types selected form the group consisting of lipstick, lip gloss, lip balm, and lip liner.

Hereinafter, preferred examples will be provided in order to illuminate one aspect of the present disclosure, however, the following examples are for illustrative purposes only, and it will be obvious to those skilled in the art that various changes and modifications may be made within the category and technological ideas of one aspect of the present disclosure, and such changes and modifications also fall within the scope of the attached claims.

In the following preparation examples, a mixed pigment composition that is a standard pigment was obtained from raw pigments through a process of color matching a No. 21 cushion foundation composition (Amorepacific).

In order to prepare the No. 21 cushion foundation composition (Amorepacific), white (W), yellow (Y), red (R), black (B) and a bulking agent (K) need to be mixed in a weight ratio of 75:5.2:1.8:0.35:17.65.

In addition, a consumer-customized cosmetic composition was color matched in the following examples by using the mixed pigment composition in a color mixing system for a cosmetic composition.

### Preparation Example 1: Preparation of Mixed Pigment Composition

Four types of pigments of white (OTS 1102 CR50, W), yellow (OTS-2 Yellow LL-100P, Y), red (OTS-2 Red R-516P, R), and black (OTS-2 Black BL-100P, B) manufactured by Daito kasei Kogyo Co., Ltd. were prepared as raw pigments. These raw pigments each correspond to a first lot by each pigment. In addition, a raw bulking agent (Ganz Chemical Co., Ltd, Ganzpearl, spherical powder, K) was prepared in order to prevent agglomeration of the raw pigments.

The four types of raw pigments and the one type of raw bulking agent were mixed to prepare four types of standard pigments as a mixed pigment composition always having constant quality, and these were each referred as standard white (MW), standard yellow (MY), standard red (MR), and standard black (MB). In addition, a bulking agent to mix with these four types of standard pigments was referred to as a standard bulking agent (MK).

MW was prepared by mixing in a ratio of 93.9% by weight of W, 0.5% by weight of Y, 0.5% by weight of R, 0.1% by weight of B, and 5% by weight of K.

MY was prepared by mixing in a ratio of 5% by weight of W, 79% by weight of Y, 5% by weight of R, 1% by weight of B, and 5% by weight of K.

MR was prepared by mixing in a ratio of 5% by weight of W, 5% by weight of Y, 79% by weight of R, 1% by weight of B, and 10% by weight of K.

MB was prepared by mixing in a ratio of 10% by weight of W, 5% by weight of Y, 5% by weight of R, 70% by weight of B, and 10% by weight of K.

As MK, the same bulking agent as K was prepared.

### Preparation Example 2: Preparation of Pigment Formulation for Color Matching

The four types of standard pigments that are the mixed pigment composition prepared in Preparation Example 1 are all in a powder form, and the mixed pigment composition having a power form was prepared to a formulation managed and mixed to be readily commercialized so that the mixed pigment composition is immediately provided to a consumer in a product form just by mixing in a cosmetics store.

In the preparation example, the mixed pigment composition having a powder form was prepared to an emulsion formulation among formulations to be commercialized by mixing, and this was referred to as a pigment formulation for color matching.

Using the composition as described in the following Table 1, the mixed pigment composition having a powder form was prepared to an emulsion formulation. Herein, four white, yellow, red and black pigment formulations for color matching were prepared respectively using standard white (MW), standard yellow (MY), standard red (MR) and standard black (MB), which are the four types of standard pigments, as the mixed pigment composition of Preparation Example 1 of the following Table 1, and these were respectively referred to as a white emulsion (E-MW), a yellow emulsion (E-MY), a red emulsion (E-MR), and a black emulsion (E-MB).

In addition, a colorless pigment formulation for color matching was prepared by mixing without using the mixed pigment composition of Preparation Example 1 in the following Table 1, and this was referred to as a colorless emulsion (E-MT).

TABLE 1

| | Content |
|---|---|
| Ingredient | (% by Weight) |
| Ethylhexyl Methoxycinnamate | 6.0 |
| Phenyl Trimethicone | 6.0 |
| Acrylates/Ethylhexyl | |
| Acrylate/Dimethicone Methacrylate | 3.0 |
| Copolymer*Cyclopentasiloxane | |
| Lauryl PEG-9 Polydimethylsiloxyethyl | 3.0 |
| Dimethicone | |
| BUTYLENE GLYCOL | 3.0 |
| DICAPRYLATE/DICAPRATE | |
| Cyclopentasiloxane*Cyclohexasiloxane | 15.3 |
| Disteardimonium Hectorite | 0.3 |
| Mixed Pigment Composition of | 10 |
| Preparation Example 1 | |
| (PF1, Function) MICRO TITANIUM | 4.0 |
| DIOXIDE MT-100TV | |
| (Function, PF1) Z-COTE HP1 | 6.0 |
| SUNPU-170 WATER | To 100 |
| Phenoxyethanol | 0.1 |
| Ethylhexyl Glycerin | 0.3 |
| SODIUM CHLORIDE | 0.1 |

### Example 1

The white emulsion (E-MW), the yellow emulsion (E-MY), the red emulsion (E-MR), the black emulsion (E-MB), and the colorless emulsion (E-MT), which are the pigment formulations for color matching prepared in Preparation Example 2, were prepared.

Using a camera that is a skin measurement module, a skin color of a person subject to skin measurement (30 years old, female) was measured, and Lab values that are skin color data were obtained.

During the camera measurement, a standard color chip was photographed at the same time to correct color variations caused by the surrounding environment.

After inputting the Lab values to a control module, a mixing ratio of the pigment formulations for color matching was obtained through a simulation of which mixing ratio of the pigment formulations for color matching needs to be used to prepare a cosmetic composition having a color matching the Lab values. Herein, color changes depending on the combination of the 5 color composition modules were databased (DB) in advance to enable the simulation.

Depending on the mixing ratio, the white emulsion (E-MW), the yellow emulsion (E-MY), the red emulsion (E-MR), the black emulsion (E-MB), and the bulking agent emulsion (E-MK), which are the pigment formulations for color matching, were mixed in a mix module to prepare a cosmetic composition having an emulsion formulation.

Herein, the Lab values were L=74.7, a=11.73 and b=20.06, respectively, and a mixing ratio of the simulated pigment formulations for color matching was E-MW:E-MY:E-MR:E-MB:E-MK=7.37:1.12:0.1:0.08:1.32.

### Example 2

Example 2 was conducted in the same manner as in Example 1, except that a cosmetic composition having an emulsion formulation was prepared using a different person subject to skin measurement (30 years old, male).

Herein, the Lab values were L=64.15, a=13.43 and b=22.41, respectively, and a mixing ratio of the simulated pigment formulations for color matching was E-MW:E-MY:E-MR:E-MB:E-MK=5.12:22.91:4.06:1.54:21.49.

### Comparative Example 1

A No. 21 cushion foundation composition (Amorepacific) was prepared.

### Experimental Example 1

On 20 test subjects having similar Lab values (error range ±0.1) to the person subject to skin measurement (30 years old, female) of Example 1, a formulation matched to each skin in the same manner as the cosmetic composition prepared in Example 1 and the No. 21 cushion foundation composition of Comparative Example 1 were used to the person subject to skin measurement (30 years old, female) of Example 1 for 7 days.

As a result, 20 of the test subjects considered that the customized cosmetic composition prepared as in Example 1 was suitable for the skin colors of themselves, whereas only 12 of the test subjects considered that the No. 21 cushion foundation composition of Comparative Example 1 was suitable for the skin colors of themselves.

Hereinbefore, one aspect of the present disclosure has been described with reference to limited examples and drawings, however, one aspect of the present disclosure is not limited thereto, and by those skilled in the art, various changes and modifications may be made within technological ideas of one aspect of the present disclosure and the range of equivalents of the attached claims.

### REFERENCE NUMERAL

1: Composition Color Mixing System
10: Skin Measurement Module
20: Control Module
30: Mix Module

1. A color mixing system for a cosmetic composition, the system comprising:
(a) a skin measurement module measuring a skin color of a person subject to skin measurement;
(b) a control module simulating, based on data on the measured skin color, a mixing ratio of pigment formulations for color matching capable of obtaining the skin color; and
(c) a mix module mixing the pigment formulations for color matching according to the simulated mixing ratio.

2. The color mixing system for a cosmetic composition of claim 1, wherein the skin measurement module is one or more types selected from the group consisting of a camera, a visible light spectrum measurement device, and a color difference meter.

3. The color mixing system for a cosmetic composition of claim 1, wherein the data are one or more types selected from the group consisting of Lab values, RGB values, and spectrum values for each wavelength for a skin color.

4. The color mixing system for a cosmetic composition of claim 1, wherein the pigment formulation for color matching is a pigment formulating a mixed pigment composition prepared by mixing raw pigments to a cosmetic composition form.

5. The color mixing system for a cosmetic composition of claim 4, wherein the raw pigment is one or more types selected from the pigment group consisting of white (W), yellow (Y), red (R), black (B), green (G), blue (B), and pink (P).

6. The color mixing system for a cosmetic composition of claim 4, wherein the mixed pigment composition is a standard pigment prepared by mixing the raw pigments and a raw bulking agent, and a standard bulking agent.

7. The color mixing system for a cosmetic composition of claim 4, wherein the standard pigment and the standard bulking agent, which are the mixed pigment composition, are mixed using a defined mixing ratio for color matching.

8. The color mixing system for a cosmetic composition of claim 1, wherein the pigment formulation for color matching has one or more types of formulations selected from the group consisting of an emulsion, a cream, an oil, a gel, a balm, and an essence.

9. The color mixing system for a cosmetic composition of claim 4, wherein the raw pigment uses four types of raw pigments of white (W), yellow (Y), red (R), and black (B) when color matching a base make-up cosmetic composition;
a color matching process is conducted by deriving MW, MY, MR, MB, and MK values using an inverse matrix in the following Equation 1; and
for each repeated color matching process, types and mixing ratios of the mixed raw pigments are derived based on MW, MY, MR, MB, and MK:
$$( \begin{matrix}{W}_{W} & {W}_{Y} & {W}_{R} & {W}_{B} & 0 \\ {Y}_{W} & {Y}_{Y} & {Y}_{R} & {Y}_{B} & 0 \\ {R}_{W} & {R}_{Y} & {R}_{R} & {R}_{B} & 0 \\ {B}_{W} & {B}_{Y} & {B}_{R} & {B}_{B} & 0 \\ {K}_{W} & {K}_{Y} & {K}_{R} & {K}_{B} & 100\end{matrix} ) \times ( \begin{matrix}MW \\ MY \\ MR \\ MB \\ MK\end{matrix} ) = ( \begin{matrix}W \\ Y \\ R \\ B \\ K\end{matrix} ) \qquad [ Equation 1 ]$$
in Equation 1,
W, Y, R, B, and K respectively mean amounts of four types of raw pigments of white, yellow, red, and black and one type of raw bulking agent used;
MW, MY, MR, MB, and MK respectively mean, as a mixed pigment composition, amounts of four types of standard pigments of white, yellow, red, and black and one type of standard bulking agent used;
W_{W}, Y_{W}, R_{W}, B_{W}, and K_{W} respectively mean mixing ratios of white, yellow, red, and black raw pigments and a raw bulking agent used for forming a white standard pigment (MW);
W_{Y}, Y_{Y}, R_{Y}, B_{Y}, and K_{Y} respectively mean mixing ratios of white, yellow, red, and black raw pigments and a raw bulking agent used for forming a yellow standard pigment (MY);
W_{R}, Y_{R}, R_{R}, B_{R}, and K_{R} respectively mean mixing ratios of white, yellow, red, and black raw pigments and a raw bulking agent used for forming a red standard pigment (MR); and
W_{B}, Y_{B}, R_{B}, B_{B}, and K_{B} respectively mean mixing ratios of white, yellow, red, and black raw pigments and a raw bulking agent used for forming a black standard pigment (MR).

10. The color mixing system for a cosmetic composition of claim 9, wherein W_{W}, Y_{W}, R_{W}, B_{W}, and K_{W} are respectively from 70 to 96, from 0.1 to 10, from 0.1 to 10, from 0.01 to 5 and from 3 to 7;
W_{Y}, Y_{Y}, R_{Y}, B_{Y}, and K_{Y} are respectively from 1 to 30, from 60 to 95, from 1 to 30, from 0.1 to 15 and from 1 to 15;
W_{R}, Y_{R}, R_{R}, B_{R}, and K_{R} are respectively from 1 to 30, from 1 to 30, from 60 to 95, from 0.1 to 15 and from 1 to 15; and
W_{B}, Y_{B}, R_{B}, B_{B}, and K_{B} are respectively from 1 to 40, from 1 to 20, from 1 to 20, from 40 to 90 and from 1 to 15.

11. The color mixing system for a cosmetic composition of claim 9, wherein the cosmetic composition is selected from the group consisting of a base make-up cosmetic composition, an eye make-up cosmetic composition, and a lip make-up cosmetic composition.

12. The color mixing system for a cosmetic composition of claim 1, wherein the bulking agent includes one or more types selected from the group consisting of polymethyl methacrylate (PMMA), silica, polymethylsilsesquioxane, an HDI/trimethylol hexyllactone crosspolymer, alumina, talc, sericite, and boron nitride.

13. The color mixing system for a cosmetic composition of claim 1, wherein the mix module includes a mixer for mixing the pigment formulations for color matching.
